(19) United States
(12) Patent Application Publication
(10) Pub. No.: US 2003/0050608 A1
(43) Pub. Date: Mar. 13, 2003

(54) Passive fluid collection device

(76) Inventor: Robert Brown, Algonquin, IL (US)

Correspondence Address: Mark S. Leonardo, Esq., Brown Rudnick Freed & Gesmer, Box IP, 18th Floor, One Financial Center, Boston, MA, 02111, US

(21) Appl. No.: 09/950,950

(22) Filed: Sep. 12, 2001

(51) Int. Cl. A61M 5/32

(52) U.S. Cl. 604/198; 600/576

(57) ABSTRACT

A fluid collection device is provided having a first cylinder and a second cylinder slidably supported by the first cylinder. At least a portion of the first cylinder flexibly engages the second cylinder to retard relative axial motion of the cylinders. A needle may be mounted to an inner surface of the first cylinder. Upon flexible engagement, the cylinders may contact in a sliding frictional engagement to retard axial motion. The first cylinder may include at least one flexible tab.

### BACKGROUND

1. Technical Field

The present disclosure generally relates to the field of fluid collection holders employed with fluid collection tubes, and more particularly, to a blood collection device configured to prevent hazardous exposure to a needle.

2. Description of the Related Art

Medical needles are well-known for injecting fluids into, or drawing blood or other fluids out of a body. During these procedures, medical needles can be exposed to the AIDS virus or any number of infectious diseases, contaminates, etc., which can present serious safety hazards to practitioners, due to accidental contact with the medical needles. These types of dangers are particularly evident during conventional blood collection procedures, such as, for example, venipuncture, to draw blood into a blood collection tube, such as test tubes, etc.

Known blood collection devices typically include a double-ended cannula or needle mounted within a barrel of a blood collection holder via a needle hub assembly. The needle hub assembly has a distal needle portion extending in one direction that is normally covered prior to use. A proximal needle portion extends in the other direction that is covered by a thin rubber membrane. During a blood collection procedure, the cover is removed from the distal needle portion and the needle is inserted into a patient's vein. An evacuated tube with a rubber stopper engages the proximal needle portion and the vacuum tube draws blood into the tube. Blood flows through the double-ended needle into the test tube and can be repeated for several blood collection tubes. The test tubes are removed from the blood collection holder and the needle is then removed from the patient. Upon removal, the needle is contaminated and potentially very dangerous. It becomes a hazardous transmission vehicle for infectious diseases.

To prevent accidental needle sticks various attempts have been made to reduce the associated hazards during a blood collection procedure. For example, the distal needle portion may be capped with a protective guard or manually retracted within the blood collection holder. However, these devices require the practitioner to use both hands to implement their protective components. Further, these designs are relatively complicated and time consuming in use.

Other devices use shield arrangements that are moved over the contaminated needle once it has been removed from the patient. However, these shield arrangements also require the use of two hands to move the shield over the contaminated needle.

Still other designs have attempted to reduce the shortcomings of the prior art by providing needle covers actuated during a blood collection procedure. These designs, however, require elaborate camming arrangements and rotation of the shield. See, e.g., U.S. Pat. Nos. 5,415,645, 5,718,239 and 5,893,845. Also, the above devices may not provide uniform and reliable motion due to their complicated cam arrangements that can jam or move out of alignment. This results in faulty operation, two-handed use and a dangerous condition to the practitioner, thereby defeating the intended purpose. These devices also require complicated molds for manufacture resulting in high production costs.

Therefore, it is desirable to have a fluid collection device that overcomes the disadvantages of the prior art by preventing hazardous exposure to a needle cannula via an actuated sheath having controlled axial motion. The fluid collection device may facilitate guided axial motion of the sheath to provide dependable performance and increased safety to the user.

### SUMMARY

Accordingly, a fluid collection device is disclosed for use with evacuated fluid collection tubes and double-ended fluid collection needle cannulas for drawing blood and/or fluids from a body. The fluid collection device prevents hazardous exposure to the double-ended needle cannula. This and other advantages are accomplished via an actuated sheath having controlled axial motion. The fluid collection device can guide axial motion of the sheath to provide dependable performance and increased safety to a practitioner during a fluid collection procedure.

In one particular embodiment, a blood collection device is provided, in accordance with the principals of the present disclosure. The blood collection device includes a first cylinder and a second cylinder slidably supported by the first cylinder. At least a portion of the first cylinder flexibly engages the second cylinder to retard relative axial motion of the cylinders. A needle may be mounted to an inner surface of the first cylinder. This configuration advantageously provides drag control of the axial motion of the components of the blood collection device.

The second cylinder may be biased between a retracted position and an extended position. The second cylinder may include at least one extension being engageable to urge the second cylinder from the retracted position. Upon flexible engagement, the cylinders may contact in a sliding frictional engagement to retard axial motion. As the first cylinder flexibly engages the second cylinder, the first cylinder can bias toward the second cylinder increasing friction therebetween. The first cylinder may include a holder disposed about the second cylinder to retard relative axial motion of the second cylinder. The holder may include a barrel. The holder may bias toward the second cylinder in a sliding frictional engagement to retard relative axial motion of the second cylinder. The first cylinder may alternatively include a sheath disposed within the second cylinder to retard relative axial motion of the sheath. The sheath may bias toward the second cylinder in a sliding frictional engagement to retard relative axial motion of the second cylinder.

In another embodiment, the needle includes a needle hub having a hub retention bead disposed about at least a portion thereof. The needle hub being releasably engageable with the second cylinder in the retracted position. Alternatively, the second cylinder includes a retention bead disposed about a distal end thereof which is releasably engageable with the first cylinder. The retention bead may releasably engage a hub retention bead of the needle hub. The second cylinder may be releasably engageable with the first cylinder in the retracted position.

In an alternate embodiment, the at least a portion of the first cylinder includes at least one flexible tab. The first cylinder may include a pair of flexible tabs diametrically disposed about the holder. The second cylinder may define an outer surface including at least one or a plurality of axial ribs. The first cylinder may define at least one slot configured for receipt of at least a portion of the axial rib. The first cylinder may include at least one flexible ring disposed about an inner surface of the first cylinder.

The first cylinder may include a flange disposed about at least a portion of an interior surface thereof. The flange can include spaced-apart undercuts. The second cylinder may include a flange disposed about at least a portion thereof such that in the extended position, the flange engages a distal end of the first cylinder to prevent axial movement of the second cylinder. The second cylinder can engage a distal end of the needle to lock the second cylinder in the extended position.

In another alternate embodiment, the blood collection device includes a first cylinder and a second cylinder slidably supported by the first cylinder and biased toward an extended position. An opposing spring is disposed between the first cylinder and the second cylinder. The opposing spring biases the second cylinder toward a retracted position. The bias of the second cylinder, toward the extended position, overcomes the bias of the opposing spring. The bias of the second cylinder toward the extended position and the bias of the opposing spring cooperate to retard relative axial motion of the cylinders. These features of the present disclosure advantageously facilitate a safe collection of body fluids and prevent inadvertent needle stick of a practitioner.

A method for collecting blood is also provided. The method includes the steps of: providing a blood collection device, similar to those described; engaging a distal end of a second cylinder to release the second cylinder from a retracted position; retarding axial movement of the second cylinder; performing blood collection; and locking the second cylinder in an extended position.

### BRIEF DESCRIPTION OF THE DRAWINGS

The objects and features of the present disclosure, which are believed to be novel, are set forth with particularity in the appended claims. The present disclosure, both as to its organization and manner of operation, together with further objectives and advantages, may be best understood by reference to the following description, taken in connection with the accompanying drawings, wherein:

FIG. 1 is a cross-sectional view of one embodiment of a blood collection device, in accordance with the principals of the present disclosure;

FIG. 2 is a perspective view of the blood collection device shown in FIG. 1 with parts separated;

FIG. 3 is a plan view of the blood collection device shown in FIG. 1;

FIG. 4 is a cross-sectional view of an indicated area of detail in FIG. 5;

FIG. 5 is an alternate side cross-sectional view of the blood collection device shown in FIG. 1, in the retracted position, including a blood collection tube;

FIG. 6 is a cross-sectional view of an indicated area of detail in FIG. 5;

FIG. 7 is a side cross-sectional view of an alternate embodiment of the blood collection device shown in FIG. 1, depicting a cap in cutaway;

FIG. 8 is a cutaway cross-sectional side view of another alternate embodiment of the blood collection device shown in FIG. 1;

FIG. 9 is a cross-sectional view of the blood collection device shown in FIG. 5 in the extended position, with the cap removed;

FIG. 10 is a cross-sectional view of an indicated area of detail in FIG. 1;

FIG. 11 is a side cross-sectional view of an alternate embodiment of the blood collection device shown in FIG. 1, in the retracted position, with the cap removed and holder in cutaway;

FIG. 12 is a side cross-sectional view of the blood collection device shown in FIG. 11;

FIG. 13 is a side cross-sectional view of the blood collection device shown in FIG. 11, in the extended position;

FIG. 14 is a side view of a sheath of the blood collection device shown in FIG. 11;

FIG. 15 is an alternate side view of the sheath shown in FIG. 14;

FIG. 16 is a cutaway cross-sectional view of an alternate embodiment of the blood collection device shown in FIG. 11;

FIG. 17 is a side cross-sectional view of an alternate embodiment of the blood collection device shown in FIG. 11;

FIG. 18 is a side cross-sectional view of another alternate embodiment of the blood collection device shown in FIG. 11;

FIG. 19 is a side cross-sectional view of a blood collection device, in the retracted position, in accordance with the principles of the present disclosure;

FIG. 20 is a side cross-sectional view of the blood collection device shown in FIG. 19, in the extended position;

FIG. 21 is a side cross-sectional view of a blood collection device, in the retracted position, in accordance with the principles of the present disclosure; and

FIG. 22 is a side cross-sectional view of the blood collection device shown in FIG. 21, illustrating a sheath in cutaway, in the extended position.

### DETAILED DESCRIPTION OF THE EXEMPLARY EMBODIMENTS

The exemplary embodiments of the fluid collection device and methods of operation disclosed are discussed in terms of fluid collection procedures, and more particularly, in terms of blood collection holders employing a double-ended needle cannula that prevent hazardous exposure to the needle cannula, including, for example, inadvertent needle stick. It is contemplated that the needle cannula may be shielded during use including storage, transport, fluid collection, subsequent to fluid collection, etc. It is envisioned, however, that the present disclosure finds application to a wide variety of cannula needles and apparatus for collection of body fluids, including, those employed during procedures relating to phlebotomy, dental, orthopedic, digestive, intestinal, urinary, veterinary, etc. It is also envisioned that the present disclosure finds application to the injection of preventive medications, medicaments, etc. to a subject.

In the discussion that follows, the term “proximal” refers to a portion of a structure that is closer to a practitioner, and the term “distal” refers to a portion that is further from the practitioner. As used herein, the term “subject” refers to a patient that receives injections or has blood and/or fluid collected therefrom using the blood collection device. According to the present disclosure, the term “practitioner” refers to an individual administering an injection, performing fluid collection, installing or removing a needle cannula from a fluid collection apparatus and may include support personnel.

The following discussion includes a description of the blood collection device, followed by a description of the method of operating the blood collection device in accordance with the present disclosure. Reference will now be made in detail to the exemplary embodiments of the disclosure, which are illustrated in the accompanying figures.

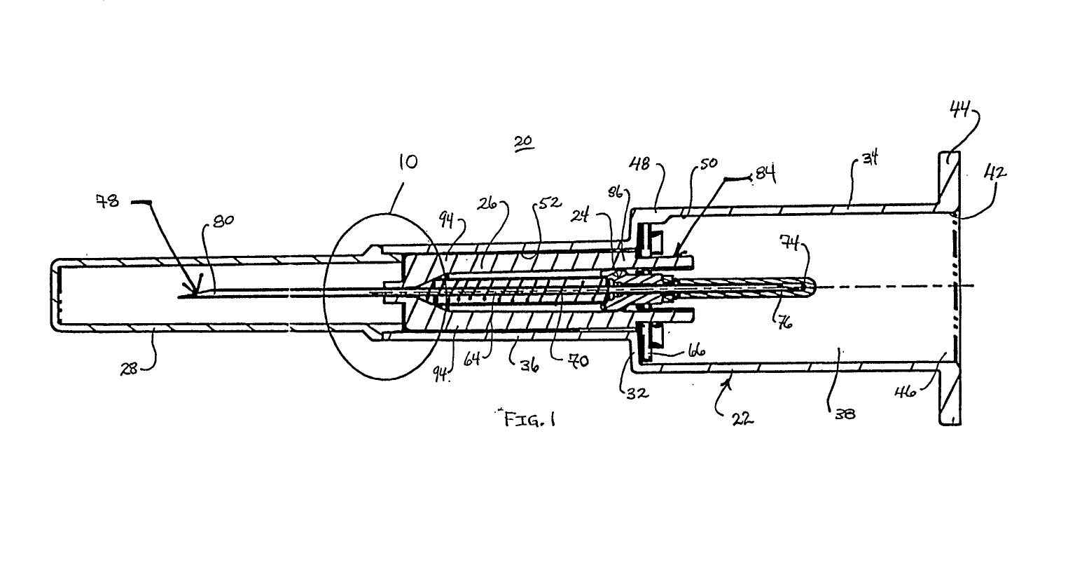

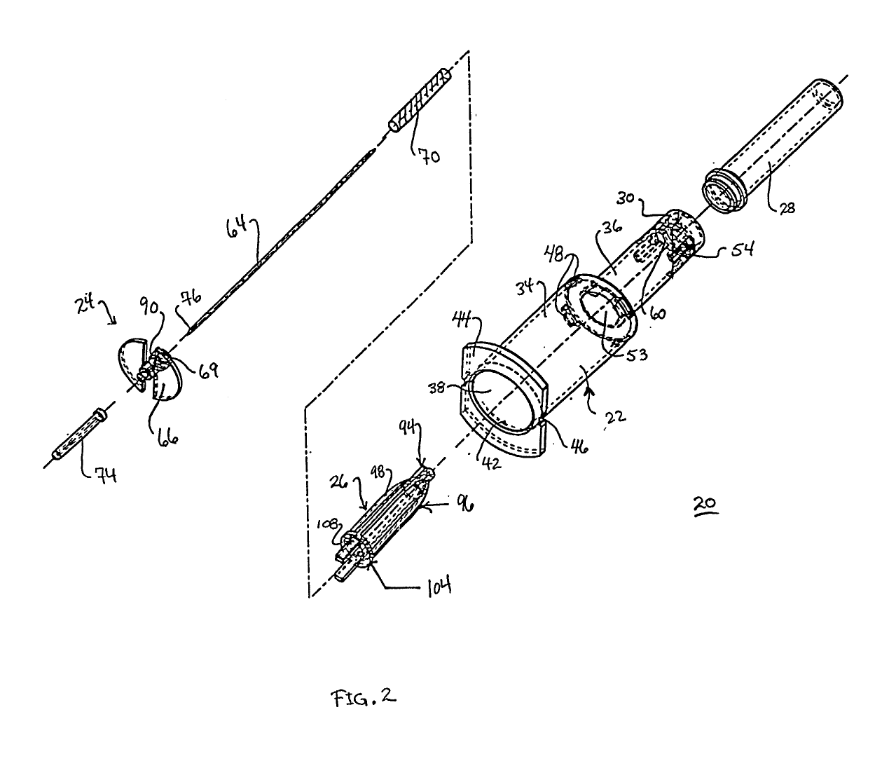

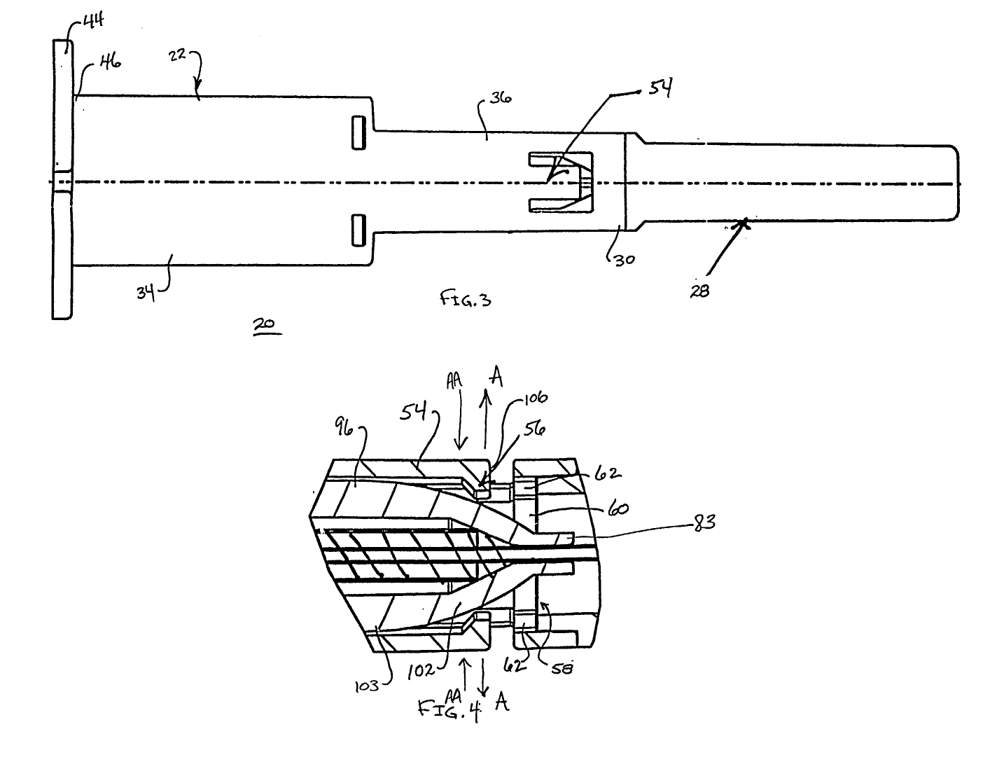

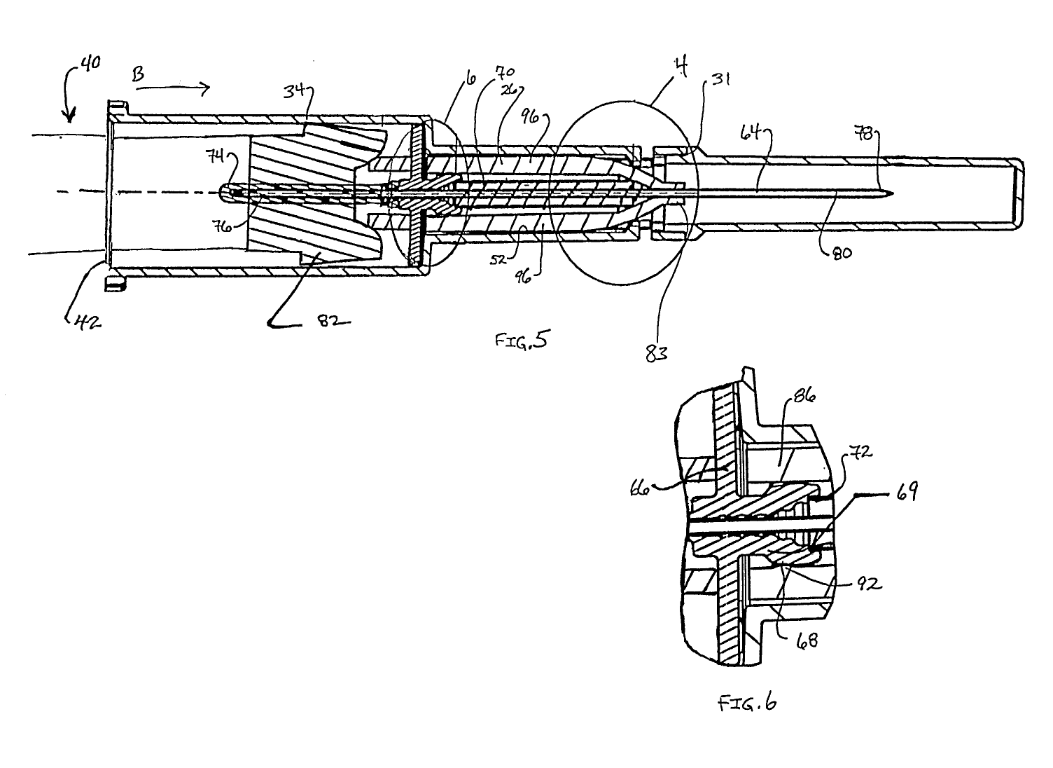

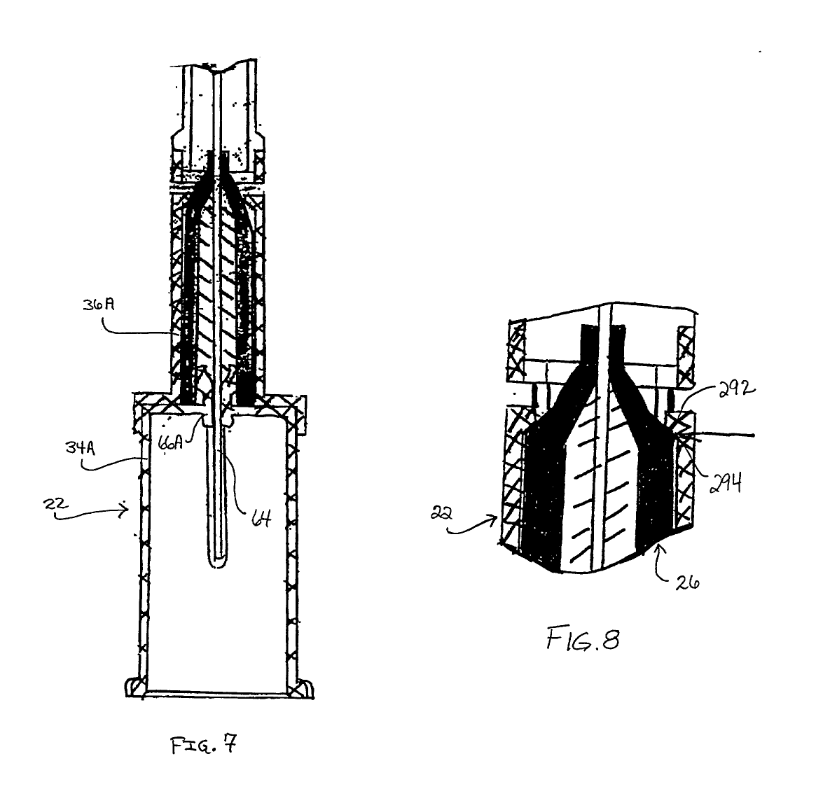

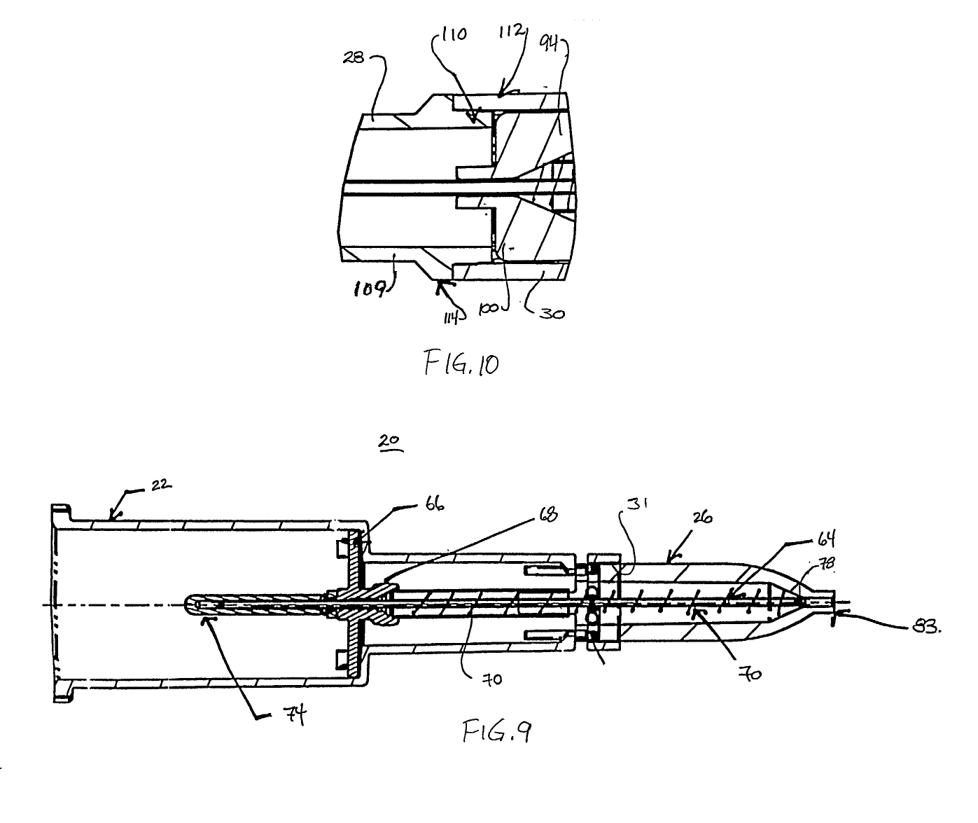

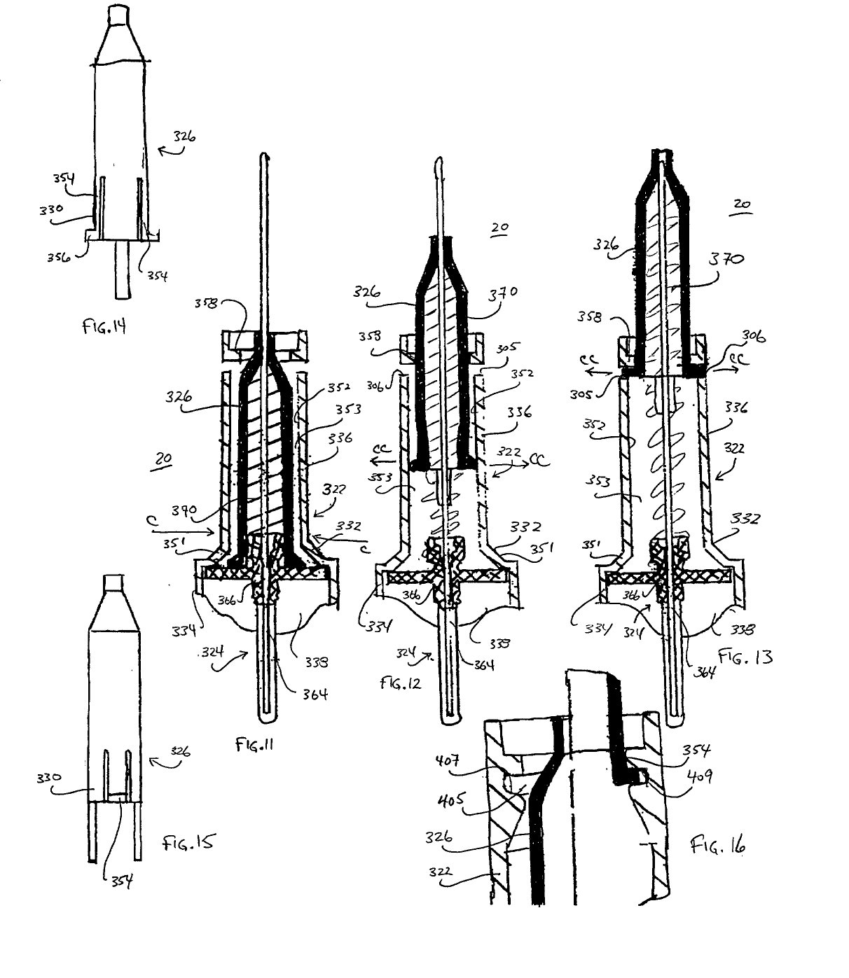

Turning now to the figures where in like components are designated by like reference numerals throughout the several views. Referring initially to FIGS. 1-3, there is illustrated a blood collection device 20, constructed in accordance with the principals of the present disclosure, including a first cylinder, such as, for example, a holder 22, a needle such as, for example, a needle assembly 24, a second cylinder such as, for example a sheath 26 and a cap 28 which engages a distal end 30 of holder 22. Blood collection device 20 is advantageously configured to prevent hazardous exposure to a needle cannula during a blood collection procedure by controlling axial motion of sheath 26 via flexible engagement with holder 22, as will be discussed below.

Sheath 26 is slidably supported within holder 22 for axial movement of sheath 26 between a retracted position and an extended position, as will be discussed. At least a portion of holder 22 flexibly engages sheath 26 to retard relative axial motion of holder 22 and sheath 26. As will be illustrated in some of the alternate embodiments disclosed herein, at least a portion of the sheath may flexibly engage the holder during relative axial motion. It is contemplated that sheath 26 may comprise the first cylinder and holder 22 may comprise the second cylinder, or that sheath 26 is disposed about an outer surface of holder 22. Accordingly, sheath 26 may be slidably supported by holder 22 or holder 22 may be slidably supported by sheath 26.

Blood collection device 20, manufactured by Kendall Healthcare Products of Mansfield, Mass. is contemplated for use in the field of blood collection. More particularly, Kendall's blood collection device 20 is envisioned to be a single use, disposable blood collection device employing, among other things, safety features having shielding capabilities to prevent inadvertent sticking or punctures of medical personnel, one-handed operation, uniform and dependable movement of sheath 26 during a procedure and a locking mechanism for reliable use. The above advantages, among others, realized from the present disclosure are attained through the disclosed blood collection device 20, which controls axial motion of sheath 26, facilitating uniform and dependable movement thereof, as discussed hereinbelow. These features of the present disclosure advantageously facilitate a safe collection of body fluids and prevent inadvertent needle stick of a practitioner.

Holder 22 includes a barrel 32, which is substantially tubular and fabricated from a material suitable for fluid collection applications, such as, for example, polymerics or metals, such as stainless steel, depending on the particular medical application and/or preference of a practitioner. Semi-rigid and rigid polymerics are contemplated for fabrication, as well as resilient materials, such as molded medical grade polypropylene. However, one skilled in the art will realize that other materials and fabrication methods suitable for assembly and manufacture, in accordance with the present disclosure, also would be appropriate. Holder 22 may be monolithically formed or, alternatively, integrally assembled of its constituent parts.

Barrel 32 has a proximal section 34 and a distal section 36. Proximal section 34 has an enlarged diameter relative to distal section 36 and a longitudinal passage 38 configured for receipt of a blood collection tube 40 (FIG. 5). Longitudinal passage 38 may have variously configured cross-sections, such as, for example, cylindrical, rectangular, etc., according to the particular medical application. Proximal section 34 may also be configured for receipt of other articles, such as, for example, syringes, etc. It is contemplated that distal section 36 has an enlarged diameter relative to proximal section 34. Proximal section 34 and distal section 36 are monolithically formed but may alternatively be integrally assembled by, for example, welding, fusion, adhesives, etc.

Proximal section 34 includes an open end 42, configured for receipt of blood collection tube 40. A flange 44 is formed adjacent a proximal end 46 of holder 22, to provide stability during operation. Holder 22 may also be constructed without flange 44. A plurality of protuberances 48 are circumferentially formed about an inner surface 50 of proximal section 34. Protuberances 48 are disposed adjacent the diameter transition between proximal section 34 and distal section 36, to facilitate mounting of needle assembly 24 to holder 22, as will be discussed below.

Distal section 36 includes an interior surface 52 and an interior passage 53 configured to support sheath 26. Interior passage 53 may have variously configured cross-sections, such as, for example, cylindrical, rectangular, etc., according to the particular configuration of sheath 26 and/or medical application. It is contemplated that proximal section 34 and distal section 36 may have varying relative diameters or, alternatively, may have a uniform diameter. It is further contemplated that holder 22 may have varying cylindrical wall thicknesses or varying dimensions of length according to the particular fluid collection application.

Distal section 36 has a pair of flexible tabs 54 diametrically disposed and formed adjacent distal end 30 of holder 22. Tabs 54 are formed in substantially parallel alignment with the cylindrical wall of distal section 36. Referring to FIG. 4, tabs 54 project from the cylindrical wall of distal section 36 in a cantilevered configuration and extend to a ramp portion 56 configured to engage sheath 26. Tabs 54 are configured to flexibly engage sheath 26 to retard relative axial motion of sheath 26. Tabs 54 flexibly engage sheath 26 such that tabs 54 are caused to flex and move relative to a surface of sheath 26, discussed below. As tabs 54 flex, they contact sheath 26 in a sliding frictional engagement to retard axial motion. This advantageously provides drag control of the axial motion of sheath 26 during a blood collection procedure.

Sliding frictional engagement of sheath 26 with ramp portion 56 causes tabs 54 to flex outwardly, in the direction shown by arrow A, from holder 22 according to the contour of sheath 26, as will be discussed below. It is contemplated that tabs 54 may variably project from the cylindrical wall of distal section 36 and/or have varying degrees of flexibility according to the particular medical application. It is further contemplated that one or a plurality of tabs 54 may be employed. It is envisioned that tabs 54 may be variously disposed along the length of distal section 36. Tabs 54 may be integrally connected to holder 22 via adhesive, clips, etc.

Distal section 36 of holder 22 has an interior flange 58 circumferentially disposed about interior surface 52. Interior flange 58 includes a plurality of spaced-apart undercuts 60 formed about interior surface 52. Undercuts 60 define slots 62 configured for slidable receipt of sheath 26, as will be discussed below, ensuring proper alignment during axial motion of sheath 26. This design beneficially facilitates guided motion of sheath 26 for uniform and reliable performance of blood collection device 20. It is contemplated that slots 62 may be of varying dimension depending on the particular medical application.

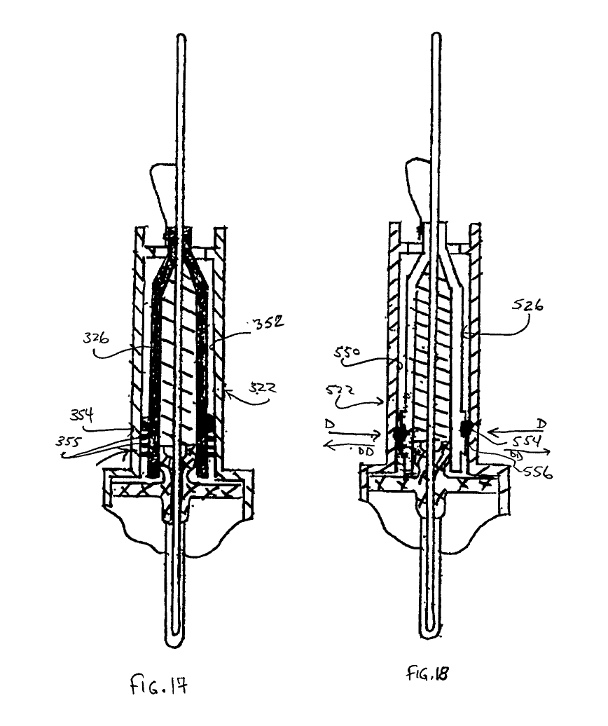

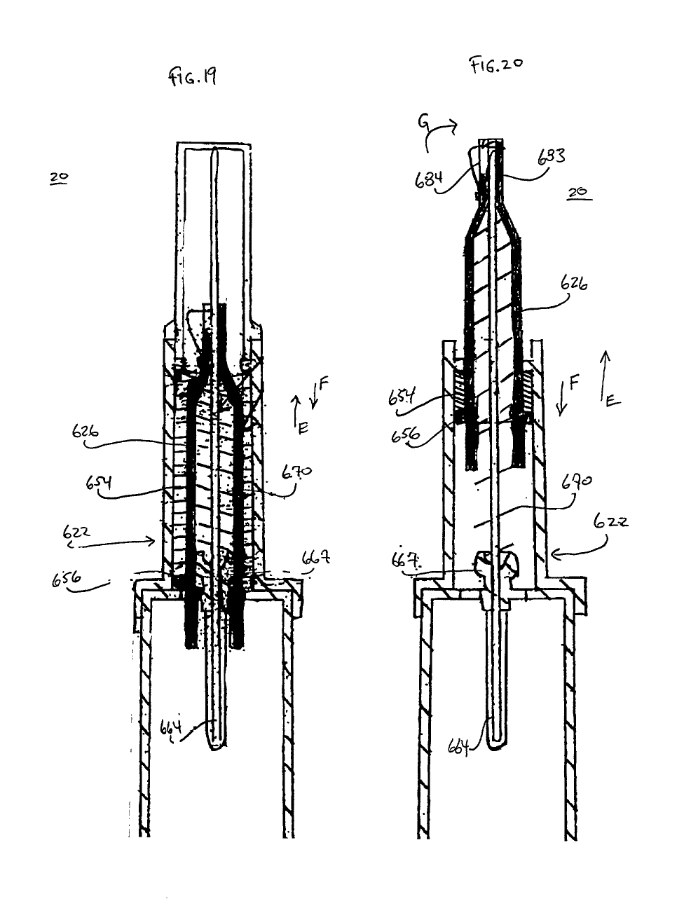

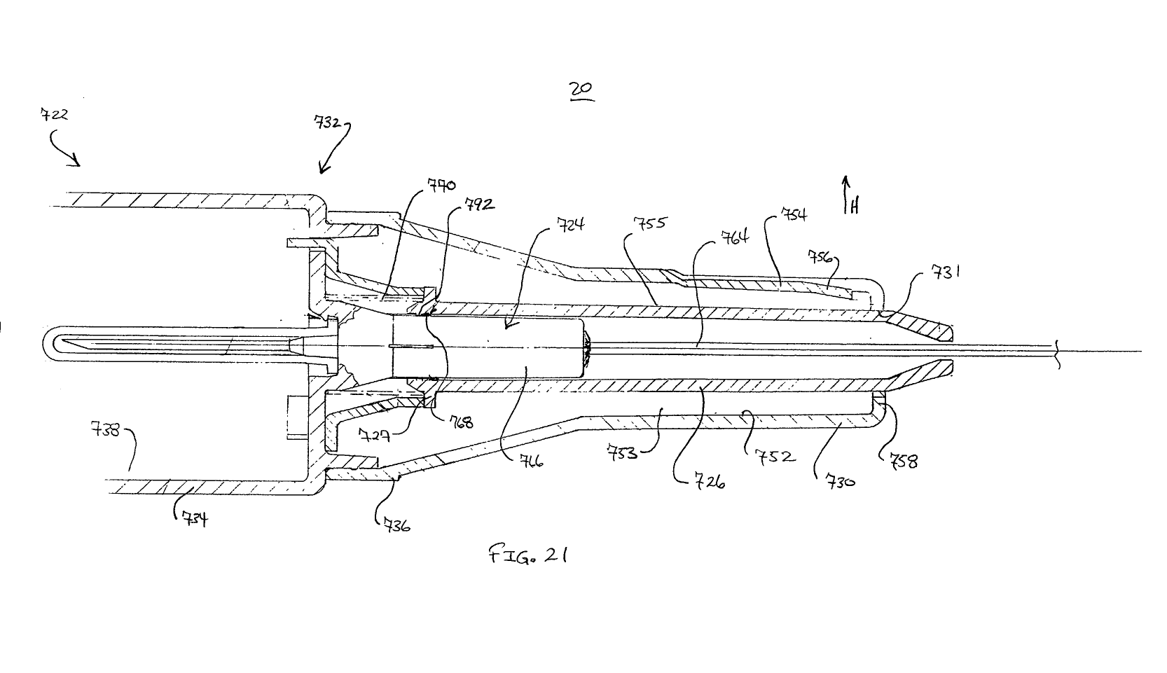

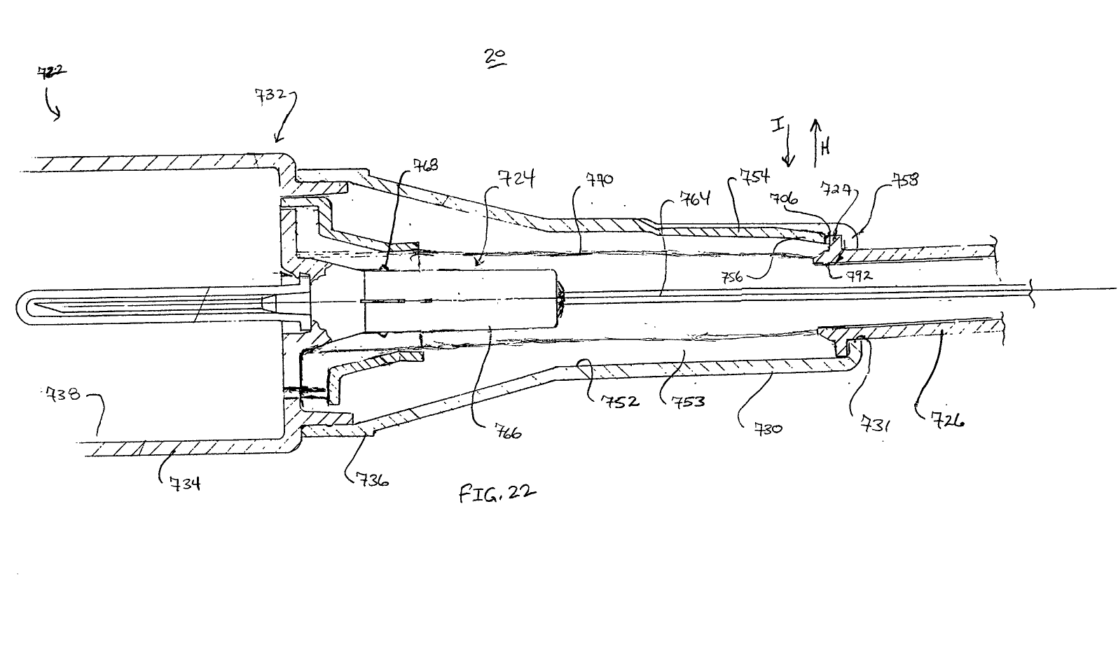

Referring back to FIGS. 1 and 2, needle assembly 24 has a needle cannula 64 and a needle hub 66 mounted therewith. Needle hub 66 mounts to inner surface 50 of holder 22 via protuberances 48 adjacent the transition diameter of proximal section 34 and distal section 36. Needle hub 66 is received within proximal section 34 and snaps over protuberances 48 to be retained thereby. It is envisioned that needle hub 66 may be mounted to holder 22 by various means including adhesives, clips, interference fit, etc.

Referring to FIGS. 5 and 6, needle hub 66 has a hub retention bead 68, disposed about a circumference of needle hub 66, for a releasable engagement with sheath 26, as will be discussed. Hub retention bead 68 may be disposed about only a portion of the circumference. Needle hub 66 includes a mounting well 69 for mounting a biasing spring 70 thereto. Biasing spring 70 is supported by needle hub 66 to bias sheath 26 from a retracted position to an extended position, as will be discussed. Mounting well 69 has a spring seat 72 configured to receive and fixedly mount a proximal end of biasing spring 70. Spring seat 72 includes grooves for threadable retention of the proximal end of biasing spring 70. Biasing spring 70 may be additionally or alternatively held within spring seat 72 by adhesives, pins, etc.

A needle membrane 74 is mounted to a proximal end 76 of needle cannula 64. Needle cannula 64 is a double-ended, blood collection needle having a needle point 78 adjacent a distal end 80 thereof. Proximal end 76 also has a sharpened tip for pierceable engagement with a stopper 82 of blood collection tube 40. It is contemplated that other cannulas which define a lumen for passage of fluids, such as, for example, syringes, etc., may be employed with blood collection device 20. In an alternate embodiment, as shown in FIG. 7, needle cannula 64 is mounted directly to holder 22. Holder 22, similar to that described above, has a distal section 36A mounted to a proximal section 34A. Proximal section 34A includes a holder hub 66A, similar to needle hub 66 described, having needle cannula 64 mounted thereto.

Sheath 26 is slideably supported within interior surface 52 of holder 22. Sheath 26 is fabricated from a material suitable for fluid collection applications, such as, for example, polymerics or metals, depending on the particular medical application and/or preference of the user. It is contemplated that the material used is a semi-rigid or rigid polymeric material suitable to enclose distal end 80 of needle 64 to prevent hazardous exposure to a practitioner. However, one skilled in the art will realize that other materials and fabrication methods suitable for assembly and manufacture, in accordance with the present disclosure, also would be appropriate.

A sheath tip 83 extends from a distal end of sheath 26 to facilitate enclosure of needle cannula 64, as will be discussed. Sheath tip 83 may extend variable lengths according to the particular medical application. As shown in FIGS. 1 and 2, sheath 26 includes a pair of sheath extensions 84 which extend from a proximal end 86 through extension slots 90 of needle hub 66. Sheath extensions 84 are engageable by stopper 82 of blood collection tube 40 for releasing sheath 26 from the retracted position (FIG. 5). It is contemplated that one or a plurality of sheath extensions 84 may be employed depending on the medical application. Sheath extensions 84 may be of variable length and may be fabricated from rigid or semi-rigid materials to facilitate separation of sheath 26 from needle assembly 24.

Sheath 26 includes a sheath retention bead 92, as shown in FIG. 6, which releasably engages hub retention bead 68 in the retracted position. Sheath retention bead 92 is disposed about a circumference of sheath 26 for corresponding engagement with hub retention bead 68. Sheath retention bead 92 and hub retention bead 68 are releasably engageable in an interference fit to provide a retaining force to maintain sheath 26 in the retracted position. The retaining force created between sheath retention bead 92 and hub retention bead 68 may be overcome upon engagement of blood tube stopper 82 for release of sheath 26 from the retracted position. It is contemplated that needle hub 66 may include one or a plurality of retention beads for engagement with corresponding sheath retention beads to maintain sheath 26 in the retracted position. It is further contemplated that other means may be used to maintain sheath 26 in the retracted position such as, for example, clips, pins, etc.

In an alternate embodiment, as shown in FIG. 8, sheath 26 includes a retention bead 292 disposed about the circumference of sheath 26 adjacent a distal end thereof. Retention bead 292 is configured for releasable engagement with a correspondingly configured retention bead 294 of holder 22, in the retracted position, similar to the bead engagement described with regard to FIG. 6.

Sheath 26 is biased via spring 70 from a retracted position, as shown in FIG. 5, to an extended position, as shown in FIG. 9. In the retracted position, sheath 26 is substantially disposed within distal section 36 of holder 22 and releasably retained thereby via engagement of hub retention bead 68 and sheath retention bead 92. Upon disengagement of beads 68 and 92, sheath 26 is biased axially toward the extended position according to the flexible and sliding frictional engagement between sheath 26 and tabs 54 of holder 22, as discussed below.

In the extended position, sheath 26 extends through an opening 31 of holder 22 beyond distal end 30, in a locked configuration with holder 22, to fully enclose needle point 78 of needle cannula 64. Sheath tip 83 encloses needle point 78, although it is envisioned that needle point 78 may be disposed within sheath 26, recessed from sheath tip 83, in the extended position. It is contemplated that spring 70 may have various degrees of resiliency, according to the requirements of a particular medical application, for biasing sheath 26 to the extended position. Alternatively, sheath 26 may be biased for axial motion by other means such as, for example, elastic bands, resilient materials, etc., or alternatively, may be fabricated from a resilient material which effectuates axial motion of sheath 26.

Sheath 26 includes a plurality of axial ribs which are slideably engageable with and disposable within grooves 62 (FIG. 4) between undercuts 60. The axial ribs include alignment/stop ribs 94 (FIG. 2) and friction ribs 96 (FIG. 5). Ribs 94, 96 are formed with an outer surface 98 of sheath 26 along the longitudinal length thereof. Ribs 94, 96 extend radially outward from sheath 26 and are appropriately dimensioned to slide through grooves 62 facilitating axially guided motion of sheath 26 relative to holder 22 during extension of sheath 26 from the retracted position (FIG. 5) to the extended position (FIG. 9). This ensures proper alignment and uniform axial movement of sheath 26 during a blood drawing procedure.

Referring to FIG. 10, alignment/stop ribs 94 have a distal stopped portion 100 configured to engage a surface of a patient (not shown) and arrest axial movement of sheath 26 at the point of contact with the surface of the patient, such as, for example, a patient's arm. Sheath 26 is biased toward the extended position, although not fully extended, upon contact with the patient's arm, to advantageously shield the needle during the blood collection procedure such that the needle is enclosed up to the point of patient contact. It is contemplated that one or a plurality of alignment/stop ribs 94 may be employed. It is further contemplated that alignment/stop ribs 94 may be variously oriented, such as, for example, angled, helical, etc. Sheath tip 83 may also engage the surface of the patient or subject.

Referring back to FIG. 4, a pair of friction ribs 96 extend along the longitudinal length of sheath 26 to an arcuate portion 102 adjacent a distal end thereof. As sheath 26 is biased toward the extended position, friction ribs 96 engage ramp portion 56 causing tabs 54 to flex in the direction shown by arrow A, on the surface of friction ribs 96. As tabs 54 flex outwardly along arcuate portion 102 to an outermost radial surface 103 of sheath 26, spring forces are created in tabs 54, in the direction shown by arrow AA. The spring forces bias tabs 54 against sheath 26 to increase friction therebetween. Thus, as tabs 54 flex, friction forces increase to retard relative axial motion of sheath 26. Advantageously, sliding frictional engagement of tabs 54 with friction ribs 96 retard axial motion of sheath 26 to provide drag control of the axial motion of sheath 26 during a blood drawing procedure. It is contemplated that tabs 54 may also flexibly and frictionally engage other portions of outer surface 98 of sheath 96 to retard axial motion of sheath 26.

Sheath 26 includes a protective flange 104 (FIG. 2) formed adjacent proximal end 86 of sheath 26. Protective flange 104 is circumferentially disposed about outer surface 98 of sheath 26. During extension of sheath 26 to the extended position, protective flange 104 is caused to engage and slide over ramp portion 56 of tabs 54 to a fixed engagement, in the extended position, with undercuts 60 of holder 22 to prevent axial movement of sheath 26. As protective flange 104 slidably engages ramp portions 56, tabs 54 are caused to flex about and slide over protective flange 104. Tabs 54 are resiliently biased to slide over protective flange 104 and come to rest in an unbiased position.

A distal face 106 of tabs 54 fixedly engages a proximal face 108 (FIG. 2) of protective flange 104 to prevent retracted axial movement of sheath 26. Protective flange 104 engages undercuts 60 to prevent extended movement of sheath 26. Distal face 106 and undercuts 60 cooperate to fixedly maintain protective flange 104 and correspondingly lock sheath 26 in the extended position to prevent hazardous exposure of needle 64 to a practitioner. It is contemplated that protective flange 104 be formed about at least a portion of outer surface 98 of sheath 26. Alternatively, sheath 26 may be releasable from the extended position.

Referring to FIG. 10, cap 28 has a proximal end 109 that includes a cap skirt 110 engaging a holder tip 112 adjacent distal end 30 of holder 22. A cap flange 114 engages holder tip 112 to provide stability of engagement of cap 28 with holder 22 and facilitate protective enclosure of needle cannula 64 during, for example, transport, storage, etc.

Referring to FIGS. 5-10, operation of blood collection device 20, similar to that disclosed, will now be described. Initially, proper preparation and sterilization of the components of blood collection device 20 is conducted. It is envisioned that preparation and sterilization may be performed before or after assembly. Prior to the blood drawing procedure, needle assembly 24 is mounted within holder 22 via hub 66, as described above. Upon assembly, sheath 26 is supported within holder 22 and needle 64 extends therethrough to needle point 78.

Sheath extensions 84 of sheath 26 extend through slots 90 of needle hub 66. Sheath 26 is maintained in the retracted position via engagement between hub retention bead 68 and sheath retention bead 92, as discussed above. During a blood drawing procedure, a practitioner (not shown) manipulates holder 22 of blood collection device 20 for operation thereof. Needle cannula 64 is inserted into a contact area (not shown) of a subject for withdrawing blood.

Referring to FIG. 5, blood collection tube 40 is inserted within open end 42 of proximal section 34, in the direction shown by arrow B. Blood tube stopper 82 of blood collection tube 40 engages sheath extensions 84 (FIG. 1) causing sheath retention bead 92 to disengage and release from hub retention bead 68. Membrane 74 allows proximal end 76 of needle cannula 64 to pass through membrane 74 and penetrate tube stopper 82. Needle cannula 64 enters the evacuated blood collection tube 40 and blood from the subject flows through needle cannula 64 into blood collection tube 40. The resulting disengagement of sheath retention bead 92 and hub retention bead 68 causes spring 70 to expand and cause sheath 26 to axially move from the retracted position (FIG. 5) towards the extended position (FIG. 9).

Alignment/stop ribs 94 slide through slots 62 during movement of sheath 26, insuring proper alignment and uniform motion of sheath 26 during the blood drawing procedure. Friction ribs 96 engage ramp portions 56 of tabs 54. Tabs 54 flex about friction ribs 96 (in the direction shown by arrows A in FIG. 4), creating friction forces in the direction shown by arrows AA, to provide drag control to the axial motion of sheath 26. Friction ribs 96 slideably engage corresponding slots 62. Drag control of the axial motion of sheath 26 is provided until sheath tip 83 and/or alignment/stop ribs 94 contact the surface of the subject for drawing blood. Upon contact with the subject surface, spring 70 remains in a partially biased position. Therefore, the apparatus and methods of the present disclosure advantageously prevent hazardous exposure to needle cannula 64 during the blood collection procedure.

After the blood drawing procedure is completed, needle tip 78 is removed from the subject's arm. From the partially biased position, spring 70 causes sheath 26 to move distally towards the extended position. Protective flange 104 slides over ramp portion 56 of tabs 54 and becomes fixed between distal face 106 of tabs 54 and undercuts 60 to lock sheath 26 in the extended position. Sheath 26 is maintained in holder 22 due to a positive interference between undercuts 60 and distal face 106 of tabs 54 on protective flange 104. Sheath tip 83 completely encloses needle point 78 to prevent hazardous exposure of needle cannula 64.

Referring to FIGS. 11-15, an alternate embodiment of blood collection device 20, similar to those described above with regard to FIGS. 1-10, is shown. Blood collection device 20 includes a sheath 326 slidably supported within a holder 322 for axial movement of sheath 326 between a retracted position (FIG. 11) and an extended position (FIG. 13) via a biasing spring 370. Holder 322 has a barrel 332 including a proximal section 334 and a distal section 336. Proximal section 334 has a longitudinal passage 338 configured for receipt of a blood collection tube (not shown). The blood collection tube engages sheath extensions of sheath 326 for release from the retracted position.

Distal section 336 has an interior surface 352 and an interior passage 353 configured for slidable support of sheath 326. An interior flange 358 is circumferentially disposed about interior surface 352. A needle assembly 324 has a needle cannula 364, and a needle hub 366. Needle hub 366 mounts to interior surface 352. Needle hub 366 is releasably engageable with sheath 326.

Referring to FIGS. 14 and 15, sheath 326 has a pair of flexible tabs 354 diametrically disposed and formed adjacent a proximal end 330 thereof. Tabs 354 are formed in substantially parallel alignment with the cylindrical wall of sheath 326. Tabs 354 project from the cylindrical wall of sheath 326 in a cantilevered configuration and extend to a ramp portion 356 configured to engage holder 322. Tabs 354 are configured to flexibly engage sheath 326 such that tabs 354 are caused to flex and move relative to surface 352 of holder 322. As tabs 354 flex, they contact holder 322 in a sliding frictional engagement to retard axial motion. Tabs 354 may be integrally connected to sheath 326 via adhesive, clips, etc.

Sliding frictional engagement of a web 351 of holder 322 with ramp portion 356 causes tabs 354 to flex inwardly, in the direction shown by arrows C, as sheath 326 is biased toward the extended position (FIG. 13). As tabs 354 flex inwardly along web 351, spring forces are created in tabs 354, in the direction shown by arrows CC (FIG. 12). The spring forces bias tabs 354 against sheath 326 to increase friction therebetween. Thus, as tabs 354 flex, friction forces increase to retard relative axial motion of sheath 326.

Upon disposal of sheath 326 in the extended position (FIG. 13), tabs 354 are biased outwardly in the direction of arrows CC, and project into an opening 305 of holder 322. In opening 305, tabs 354 fixedly engage a distal face 306 and interior flange 358 to prevent axial movement of sheath 326. The cooperative engagement of distal face 306 and interior flange 358 lock sheath 326 in the extended position. Alternatively, as shown in FIG. 16, holder 322 has an inner cavity 405 having a distal face 407 and a proximal face 409. In the extended position, tabs 354 are disposed within cavity 405 in a fixed engagement with distal face 407 and proximal face 409. The cooperative engagement of distal face 407 and proximal face 409 lock sheath 326 in the extended position.

Referring to FIG. 17, in an alternate embodiment, tabs 354 include concentric bands 355 disposed axially along sheath 326 for flexible and sliding frictional engagement with interior surface 352 of holder 322. Referring to FIG. 18, in another alternate embodiment, a sheath 526 is shown, similar to that described above. Sheath 526 includes a flexible ring 554 disposed within an outer cavity 556 and about the circumference of sheath 526. Outer cavity 556 is formed adjacent a proximal end of sheath 526 and retains rings 554 therein via adhesives, pins, clips, etc. Flexible ring 554 may also be press-fit, etc., to sheath 526. Flexible ring 554 is fabricated from an elastic polymeric material suitable for medical needle applications in accordance with the principles of the present disclosure. It is contemplated that suitable metals may also be used.

As sheath 526 translates from a retracted to an extended position, flexible ring 554 engages an inner surface 550 of a holder 522. Flexible ring 554 flexes inwardly, in the direction shown by arrows D, to flattened or distorted shape, creating spring forces therein, in the direction shown by arrows DD. The spring forces bias flexible ring 554 adjacent holder 522 to increase friction therebetween. Thus, as flexible ring 554 flexes, friction forces increase to retard relative axial motion of sheath 526.

It is envisioned that a plurality of flexible rings 554 may be used. It is further envisioned that the flexible ring may have an O-ring configuration such that a more rigid material is used and the O-ring flexes within outer cavity 586.

Referring to FIGS. 19 and 20, yet another alternate embodiment of blood collection device 20, similar to those described, is shown. Blood collection device 20 includes a sheath 626 slidably supported by a holder 622. Sheath 626 is biased from a retracted position (FIG. 19) to an extended position (FIG. 20) via a biasing spring 670. A needle cannula 664 is mounted to a holder hub 667 of holder 622.

An opposing spring 654 is mounted within holder 622 between sheath 626. A proximal end of opposing spring 654 engages a flange 656 of sheath 626. A distal end of opposing spring 654 engages a distal end of holder 622. Opposing spring 654 biases sheath 626 towards the retracted position (FIG. 19). In the retracted position, sheath 626 compresses biasing spring 670 and is releasably engaged with holder hub 667. The compression of biasing spring 670 maximizes its spring force and corresponding bias of sheath 626 towards the extended position. Opposing spring 654 is expanded thereby minimizing its spring force.

Upon release of sheath 626 from engagement with holder hub 667, sheath 626 is permitted to move toward the extended position. The spring force of biasing spring 670, in the direction shown by arrow E, is maximized, thus overcoming the minimized spring force of opposing spring 654 in the direction shown by arrow F. Therefore, sheath 626 is urged toward its extended position due to the resultant force in the extended direction. However, as sheath 626 moves axially towards the extended direction, opposing spring 654 is compressed to maximize its spring force. This cooperation of spring 654, 670 retards relative axial motion of sheath 626. The spring force of biasing spring 670 is sufficient to overcome the spring force of opposing spring 654 such that sheath 626 translates to the extended position.

A sheath tip 683 extends from the distal end of sheath 626. A latch 684 is pivotally mounted to sheath tip 683. Latch 684 pivots via a leaf spring or the like. The leaf spring biases latch 684 from an open position to a closed position, in the direction shown by arrow G. In the retracted position of sheath 626, a distal end of needle cannula 664 extends through sheath tip 683 forcing latch 684 to the open position via engagement with needle cannula 664. As sheath 626 is urged toward the extended position, needle cannula 664 is retracted such that as the distal end of needle cannula 664 passes and disengages from latch 684, latch 684 is biased to the closed position to fully enclose the distal end of needle cannula 664.

Referring to FIGS. 21 and 22, another alternate embodiment of blood collection device 20, similar to those described above, is shown. Blood collection device 20 includes a sheath 726 slidably supported within a holder 722 for axial movement of sheath 726 between a retracted position (FIG. 21) and an extended position (FIG. 22) via a biasing spring 770. Holder 722 has a barrel 732 including a proximal section 734 and a distal section 736. Proximal section 734 has a longitudinal passage 738 configured for receipt of a blood collection tube (not shown). The blood collection tube engages sheath extensions of sheath 726 for release from the retracted position.

Distal section 736 has an interior surface 752 and an interior passage 753 configured for slidable support of sheath 726. An interior flange 758 is circumferentially disposed about interior surface 752 adjacent a distal end 730 of holder 722. Interior flange 758 defines an opening 731 through which sheath 726 extends. A needle assembly 724 has a needle cannula 764, and a needle hub 766. Needle hub 766 mounts to interior surface 752.

Sheath 726 includes a sheath retention bead 792 which releasably engages a hub retention bead 768 of needle hub 766, in the retracted position (FIG. 21). Sheath retention bead 792 is disposed about a circumference of sheath 726 for corresponding engagement with hub retention bead 768. Sheath retention bead 792 and hub retention bead 768 are releasably engageable in an interference fit to provide a retaining force to maintain sheath 726 in the retracted position. The retaining force created between sheath retention bead 792 and hub retention bead 768 may be overcome upon engagement of the blood collection tube for release of sheath 26 from the retracted position.

Distal section 736 of holder 722 has a flexible tab 754 formed adjacent proximal end 730. Tab 754 is formed in substantially parallel alignment with the cylindrical wall of distal section 736. Tab 754 projects from the cylindrical wall of distal section 736 in a cantilevered configuration for engagement with sheath 726. Tab 754 is configured to flexibly engage a flange 727 of sheath 726 such that tabs 754 are caused to flex and move relative to flange 727. It is contemplated that one or a plurality of tabs 754 may be employed and variously disposed about holder 722.

As sheath 726 is released from the retracted position (FIG. 21), sheath 726 is urged towards the extended position (FIG. 22) via biasing spring 770. Sheath 726 approaches the extended position and flange 727 engages tab 754, causing tab 754 to flex outwardly, in the direction shown by arrow H. Tab 754 flexes relative to flange 727 and comes to rest such that a distal face 706 of tab 754 engages flange 727. Upon disposal of sheath 726 in the extended position (FIG. 22), flange 727 fixedly engages distal face 706 and interior flange 758 to prevent axial movement of sheath 726. The cooperative engagement of distal face 706 and interior flange 758 lock sheath 726 in the extended position Alternatively, tab 754 may be oriented within passage 753 such that as tab 754 flexes, it contacts an outer surface 755 of sheath 726 in a sliding frictional engagement to retard axial motion. Sliding frictional engagement of sheath 726 with a ramp portion 756 of tab 754 causes tab 754 to flex outwardly, in the direction shown by arrow H, as sheath 726 is biased toward the extended position (FIG. 22). As tab 754 flexes outwardly, spring forces are created in tab 754, in the direction shown by arrow I (FIG. 21). The spring forces bias tab 754 against sheath 726 to increase friction therebetween. Thus, as tab 754 flexes, friction forces increase to retard relative axial motion of sheath 726. Sheath 726 may also include friction ribs and/or alignment ribs, similar to that described above.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A fluid collection device comprising:
a first cylinder; and
a second cylinder slidably supported by the first cylinder, wherein at least a portion of the first cylinder flexibly engages the second cylinder to retard relative axial motion of the cylinders.

2. A fluid collection device according to claim 1, wherein the second cylinder is biased between a retracted position and an extended position.

3. A fluid collection device according to claim 2, further comprising a needle mounted to an inner surface of the first cylinder.

4. A fluid collection device according to claim 1, wherein upon flexible engagement, the cylinders contact in a sliding frictional engagement to retard axial motion.

5. A fluid collection device according to claim 1, wherein as the at least a portion of the first cylinder flexibly engages the second cylinder, the at least a portion of the first cylinder biases toward the second cylinder increasing friction therebetween.

6. A fluid collection device according to claim 1, wherein the first cylinder includes a holder disposed about the second cylinder to retard relative axial motion of the second cylinder.

7. A fluid collection device according to claim 1, wherein the first cylinder includes a sheath disposed within the second cylinder to retard relative axial motion of the sheath.

8. A fluid collection device according to claim 1, wherein the at least a portion of the first cylinder includes at least one flexible tab.

9. A fluid collection device according to claim 1, wherein the at least a portion of the first cylinder includes a pair of flexible tabs being diametrically disposed about the holder.

10. A fluid collection device according to claim 3, wherein the needle includes a needle hub having a hub retention bead disposed about at least a portion thereof and being releasably engageable with the second cylinder in the retracted position.

11. A fluid collection device according to claim 3, wherein the second cylinder includes a retention bead disposed about a distal end thereof which is releasably engageable with the first cylinder.

12. A fluid collection device according to claim 1, wherein the second cylinder defines an outer surface including at least one axial rib.

13. A fluid collection device according to claim 1, wherein the second cylinder defines an outer surface including a plurality of axial ribs.

14. A fluid collection device according to claim 12, wherein the first cylinder defines at least one slot configured for receipt of at least a portion of the at least one axial rib.

15. A fluid collection device according to claim 1, wherein the first cylinder includes a flange disposed about at least a portion of an interior surface thereof.

16. A fluid collection device according to claim 15, wherein the flange includes spaced-apart undercuts.

17. A fluid collection device according to claim 11, wherein the retention bead releasably engages a hub retention bead of the needle.

18. A fluid collection device according to claim 1, wherein the second cylinder includes at least one extension being engageable to urge the second cylinder from the retracted position.

19. A fluid collection device according to claim 2, wherein the second cylinder includes a flange disposed about at least a portion thereof such that in the extended position, the flange engages a distal end of the first cylinder to prevent axial movement of the second cylinder.

20. A fluid collection device according to claim 2, wherein the second cylinder is releasably engageable with the first cylinder in the retracted position.

21. A fluid collection device according to claim 3, wherein the second cylinder engages a distal end of the needle to lock the second cylinder in the extended position.

22. A fluid collection device according to claim 1, wherein the at least a portion of the first cylinder includes at least one flexible ring disposed about an inner surface of the first cylinder.

23. A fluid collection device according to claim 1, wherein the first cylinder includes a proximal section having a diameter greater than a diameter of a distal section thereof.

24. A fluid collection device comprising:
a holder;
a needle mounted to the holder; and
a sheath supported by the holder and biased between a retracted position and an extended position, wherein at least a portion of the sheath flexibly engages the holder such that the at least a portion of the sheath biases toward the holder in a sliding frictional engagement to retard relative axial motion of the sheath.

25. A fluid collection device comprising:
a holder;
a needle mounted to the holder; and
a sheath supported by the holder and biased between a retracted position and an extended position, wherein at least a portion of the holder flexibly engages the sheath such that the at least a portion of the holder biases toward the sheath in a sliding frictional engagement to retard relative axial motion of the sheath.

26. A blood collection device comprising:
a holder including a barrel and a pair of flexible tabs formed adjacent a distal end thereof, the holder including a plurality of spaced apart undercuts formed on an interior surface thereof;
a needle assembly having a needle cannula and a needle hub including a hub retention bead and being mounted to an inner surface of the holder; and
a sheath slidably supported within the holder and spring biased from a retracted position to an extended position, the sheath having a pair of sheath extensions being engageable for releasing the sheath from the retracted position, the sheath including a sheath retention bead that releasably engages the hub retention bead in the retracted position, and further including a plurality of axial ribs being disposable between the undercuts, at least one of the axial ribs flexibly engaging the pair of flexible tabs such that the flexible tabs bias toward the sheath in a sliding frictional engagement to retard axial motion of the sheath from the retracted to the extended position, the sheath further including a flange disposed adjacent a proximal end thereof that engages the undercuts of the holder to prevent axial movement of the sheath.

27. A fluid collection device comprising:
a first cylinder including flexible means;
a needle assembly including a needle hub means mounted to the first cylinder;
a second cylinder supported by the first cylinder; and
means for translating the second cylinder between a retracted position and an extended position.

28. A fluid collection device comprising:
a first cylinder;
a second cylinder slidably supported by the first cylinder and biased toward an extended position; and
an opposing spring disposed between the first cylinder and the second cylinder, the opposing spring biasing the second cylinder toward a retracted position, wherein the bias of the second cylinder toward the extended position overcomes the bias of the opposing spring.

29. A fluid collection device according to claim 28, wherein the bias of the second cylinder toward the extended position and the bias of the opposing spring cooperate to retard relative axial motion of the cylinders.

30. A method for collecting blood, the method comprising the steps:
providing a blood collection device including:
a first cylinder;
a second cylinder slidably supported by the first cylinder, wherein at least a portion of the first cylinder flexibly engages the second cylinder to retard relative axial motion of the cylinders;
engaging a distal end of the second cylinder to release the second cylinder from a retracted position;
retarding axial movement of the second cylinder;
performing blood collection; and
locking the second cylinder in an extended position.
